(19) United States
(12) Patent Application Publication
(10) Pub. No.: US 2004/0148656 A1
(43) Pub. Date: Jul. 29, 2004

(54) Transient production of pharmaceutically important proteins in plants

(75) Inventors: Valentin Negrouk, Weston, FL (US); Galina Negrouk, Weston, FL (US); Newell Bascomb, Brookside, NJ (US); Hyung Lee, Weston, FL (US); Dean Taylor, Weston, FL (US)

Correspondence Address: EDWARDS & ANGELL, LLP, P.O. BOX 55874, BOSTON, MA, 02205, US

(73) Assignee: Sunol Molecular Corporation

(21) Appl. No.: 10/739,447

(22) Filed: Dec. 17, 2003

Related U.S. Application Data
(60) Provisional application No. 60/436,403, filed on Dec. 23, 2002.

(51) Int. Cl. A01H 1/00; C12N 15/82

(52) U.S. Cl. 800/288; 800/294

(57) ABSTRACT

The invention relates to a rapid, versatile method for production of biopharmaceutical proteins and other valuable proteins in a eukaryotic system. It features an efficient and inexpensive method for transient production of monoclonal antibodies and other pharmaceutically important proteins by introduction of Agrobacterium bearing genes for the protein of interest into already grown plant hosts, followed by recovery of the protein of interest.

### RELATED APPLICATION

This application claims priority to U.S. Provisional Application, Serial No. 60/436,403, filed Dec. 23, 2002, the entirety of which is incorporated by reference herein.

### FIELD OF THE INVENTION

The invention relates to methods and kits for high-level transient protein production in plants.

### BACKGROUND OF THE INVENTION

The expression of recombinant pharmaceutically important proteins is usually performed using microbial or mammalian hosts. Microbial systems often offer advantages in speed of cloning and producing transformed cells. While yields of heterologous gene products can be typically high, the product that accumulates is often not biologically active, requiring costly and difficult re-folding to achieve active material. Also many post-translational modifications are different in bacteria compared to eukaryotes, so certain categories of proteins cannot be properly expressed in prokaryotic systems.

One of the major problems with the use of heterologous systems for expressing pharmaceutically important proteins is the time required to proceed from a cloned gene to production of a functional protein in sufficient quantity for use in a relevant animal model. With most systems, this time can be many months to a year or more. In addition to being time consuming, transient production of recombinant proteins in cell culture (e.g., such as by transient transfection of COS or CHO cells or by baculovirus infection of insect cell culture) typically requires specialized equipment and special skills are required to handle the cells and viruses necessary for such production. In addition, the amount of protein obtained is usually very small (nanogram to microgram quantities) unless a scaled-up process is implemented which can require even more expensive and specialized equipment and training.

Since the late 1980's, there have been numerous examples disclosing cost-effective expression of foreign or heterologous proteins in crop plants. In particular, plants have emerged in recent years as an expression system for production of monoclonal antibodies and other pharmaceutically important proteins. Plants have a protein synthesis pathway very similar to animal cell pathways, with differences primarily in protein glycosylation (Fischer and Emans, Transgenic Res. 9: 279-299 (2000); Cabanes-Macheteau, et al., Glycobiology 2: 365-72 (1999)). In addition, some proteins of interest have been shown to accumulate to high levels in plants. Further, plant-derived antibodies are functionally equivalent to those produced by hybridomas (Fischer and Emans, supra, (2000)). Finally, plant-derived antibodies and other proteins do not contain human or animal pathogens or co-purified blood-borne pathogens and oncogenic sequences that can accompany recombinant proteins purified in other systems (Fischer and Emans, supra (2000)).

Recombinant proteins may be produced from stably integrated genes in transgenic plants. Another option to generate protein from a heterologous gene is to use a transient expression system. Several systems have been used to develop gene cloning approaches, plasmid constructs, promoters, etc, that could be applied to this end. In particular, electroporation of protoplasts has been used extensively, as well as particle bombardment and to some degree viral vectors.

Particle bombardment usually reaches only a few cells and the DNA must reach the cell nucleus for transcription to be accomplished (Christou, Plant Mol. Biol. 35: 197-203 (1996); Fischer and Emans, supra (2000)). The use of Agrobacterium delivered by infiltration (agro-infiltration) can deliver foreign genes to significantly higher number of cells. Additionally, T-DNA harboring the gene of interest is actively transferred into the nucleus with the aid of several bacterial proteins (Kapila et al., Plant Sci. 122: 101-108 (1997); Fischer and Emans, supra (2000)). While both particle bombardment and agro-infiltration result in heterologous protein expression within 3-5 days after treatment, viral delivery takes from 2 to 4 weeks. The use of particle bombardment is not very efficient for transient expression but is much more important for regeneration of transgenic cereal crops (Christou, supra (1996); Fischer and Emans, supra (2000)).

Infection with a modified viral vector results in systemic spread of the virus throughout the most plant cells. The introduced gene is transcribed by viral RNA replicase in the cytoplasm and is translated into the protein of interest. Target genes are expressed in high levels in recombinant viral vectors because of the high level of multiplication during viral replication (Porta and Lomonossoff, Mol. Biotechnol. 5: 209-21 (1996)). However, usually this system is limited to proteins with a molecular mass of less than 60-70 Kd.

Agro-infiltration for transient expression has a number of advantages. The method produces the protein of interest within days and yields quantities of protein sufficient for characterization of protein stability and protein function (Fischer and Emans, supra (2000)). It has been proposed that agro-infiltration could be scaled up to produce tens of milligrams of recombinant proteins without the need of stably transformed plants. However, at the levels of expression reported (Fischer and Emans, supra (2000)), it would not be practical for any larger scale studies, such as in vivo animal models, where greater quantities (e.g., 10-100 mgs) of protein are needed.

The original system of Agrobacterium infiltration for transient expression was described by Kapila (Kapila et al., supra (1997)) and was developed for rapid testing of the functionality of a protein thought to be useful for disease resistance of the plant tissue. For this application the protein would not need to be purified or characterized since the entire plant tissue could be used in a bioassay. This system was later used to express pharmaceutically important proteins (Vaquero et al., Mol. Biotechnol. 5: 209-21 (1996)). A chimeric antibody against human carcinoembryogenic antigen and a recombinant single chain antibody against the same antigen was produced, purified, biochemically analyzed and shown to be similar to the animal derived protein. However, the production from this system was relatively low.

### SUMMARY OF THE INVENTION

The invention provides a substantially improved process for producing proteins in already grown, commercially available plants without the need to have plant growth facilities. The method provides a biologically functional protein very rapidly with minimal time and expense on a scale which is suitable for testing in animals, analysis in multiple assays, characterization of crystal structures, assays of protein modification, and the like. The method can be used to produce at least about 1 mg, at least about 5 mg, and at least about 10 mg of protein at a single time.

The method of the invention can be easily and readily scaled providing milligram quantities required for repeated testing and can be used to functionally evaluate pharmaceutical proteins.

The present invention provides methods and kits for transient expression of monoclonal antibodies and other pharmaceutically important proteins. This method has been particularly successful in producing proteins in lettuce that has been vacuum-infiltrated with Agrobacterium tumefaciens bearing recombinant genes of interest on plasmid vectors with or without viral regulatory sequences.

In one aspect, the invention provides a bacterial/plant hybrid expression system that can be used to produce 10-50 mg/kg of protein per kg very rapidly and is scalable. In one preferred aspect, one or more heterologous genes are delivered to a plant tissue using a disarmed or virulent strain of Agrobacterium. Preferably, the plant tissue is from an already grown plant (e.g., such as a plant obtained from a store). A particularly preferred source of plant tissue is lettuce. The method disclosed herein combines the advantage of rapid gene cloning and manipulation in a bacterial system with the advantages of protein production in a eukaryotic cell environment which provides the necessary milieu for appropriate targeting, processing, modification, and assembly without the need for growing plants or handling transgenic plant material.

In one aspect, cells of Agrobacterium bearing expression constructs with a heterologous gene or genes of interest are used to deliver the heterologous gene(s) to a plant tissue for transient expression in the cells and/or extracellular spaces of the plant tissue. Generally, a suitable expression construct comprises: at least one T-DNA border sequence, an expression control sequence (e.g., a promoter which may be inducible and/or tissue-specific, or constitutive), and a gene of interest operably linked to the expression control sequence. In one aspect, an expression construct is part of a vector comprising one or more origins of replication, at least one origin of replication suitable for replicating the vector comprising the expression construct in Agrobacterium.

Cultures of Agrobacterium cells comprising the expression construct are infiltrated into plant tissue in the presence of a surfactant. Preferably, infiltration occurs in the presence of a vacuum. After incubating the plant tissue under suitable conditions that allow the expression construct to express the protein in a plurality of plant cells, the protein is isolated from the cells. The method requires only a single round of contacting the plant tissue with Agrobacterium comprising the vector, infiltrating the plant tissue with vector and expressing the heterologous protein to obtain yields of from about 500 μg-500 mg. However, additional rounds of agro-infiltration and purification may be performed to scale up the procedure. More preferably, more plant tissue is used in a single round of the method.

The Agrobacterium used can be wild type (e.g., virulent) or disarmed. Multiple Agrobacterium strains, each expressing different genes can be used to produce the individual proteins or a heteromultimeric protein (e.g., antibody) or to reproduce a pathway, such as a metabolic pathway, a chemical synthesis pathway or a signaling pathway. Alternatively, or additionally, a single Agrobacterium strain may comprise a plurality of sequences comprising different heterologous genes. The different heterologous genes may be comprised within a single nucleic acid molecule (e.g., a single vector) or may be provided in different vectors. In one aspect, at least one Agrobacterium strain comprises Agrobacterium tumefaciens.

Because the invention provides a high throughput system for expressing heterologous proteins, the system can be use to determine the effect of variation in a gene and/or protein of interest on the function of the protein. In one aspect, to evaluate a vast number of variant heterologous proteins, a plurality of expression constructs is produced using standard molecular biology techniques in bacteria (e.g., by random mutagenesis, by combinatorial cloning techniques, and the like) comprising nucleic acids encoding proteins which are substantially identical (e.g., greater than about 50% identical, preferably greater than 75% identical, more preferably greater than about 90% or 95% identical) and which can be produced for rapid screening for biological activity using the transient expression system according to the invention. In one aspect, the plurality of expression constructs comprise, greater than about 100, greater than about 500, greater than about 1×10³, greater than about 1×10⁴, greater than about 1×10⁵, greater than about 1×10⁶, greater than about 1×10⁷, greater than about 1×10⁸, or greater than about 1×10⁹ variant encoding sequences.

The plurality of expression constructs can comprise a library of sequences comprising random or semi-random variations in the coding sequences of heterologous polypeptides. The library may be an E. coli-based library (i.e., individual library members are cloned and replicated in E. coli) or an Agrobacterium-based library (i.e., individual library members are cloned and replicated in Agrobacterium) or a combination thereof (i.e., cloning may initially be performed in E. coli and library members may be subsequently introduced into Agrobacterium cells for further replication and/or cloning). Individual members of the library are tested for polypeptide function after transient expression in a plant tissue, for example to identify polypeptides suitable for larger scale production and/or for production in transgenic plants.

In one preferred aspect, the method is used to optimize the function of interacting subunit elements (ISEs) of a multi-subunit protein complex for optimal activity and/or binding. For example, to optimize antibody binding, a plurality of variants of antibody variable regions is produced using standard molecular biology techniques in bacteria (e.g., by random mutagenesis, by combinatorial cloning techniques, and the like). Preferably, greater than about 1×10³, greater than about 1×10⁴, greater than about 1×10⁵, greater than about 1×10⁶, greater than about 1×10⁷, greater than about 1×10⁸, or greater than about 1×10⁹ variant expression constructs are produced. Suitable variable region sequences include the light chain (LC), or the heavy chain (HC), or both light and heavy chains of an antibody. Variant polypeptides comprising these sequences are evaluated for specific binding to a selected antigen. The variant polypeptides may comprise full-length antibodies or antigen-binding fragments (scFv, Fab′, etc.) thereof. The different variants are readily cloned into the Agrobacterium vectors described above and all combinations of HC and LC can be rapidly tested. In one preferred aspect, testing occurs in parallel.

The method can be used to pre-screen expression vectors most suitable for protein expression in a growing plant. In one aspect, the method is used to rapidly screen for variants of expression control sequences and/or translation control sequences which provide for optimal protein expression. Alternatively, or additionally, variant sequences are screened to identify sequences encoding proteins with increased stability or other desired pharmaceutical properties.

The invention also provides kits useful for performing the method. In one aspect, a kit according to the invention includes a cloning/expression vector suitable for expression in at least an Agrobacterium species such as A. tumefaciens, and one or more components for infiltrating, extracting and/or purifying a desired heterologous protein from a plant species. In another aspect, the kit further comprises one or more bacterial strains (e.g., such as E. coli and A. tumefaciens). In still a further aspect, the kit comprises a plurality of expression constructs comprising nucleic acids encoding variant heterologous sequences.

### BRIEF DESCRIPTION OF THE DRAWINGS

The objects and features of the invention can be better understood with reference to the following detailed description and accompanying drawings.

FIGS. 1A and 1B are diagrams of the plasmids pSUNP1 and pSUNP2 used in co-infiltration of a plant tissue according to one aspect of the invention. Plasmid pSUNP1 expresses the heavy chain of hOAT from the OCS3MAS promoter and pSUNP2 expresses the light chain. Shown are the T-DNA borders which lead to the movement of all genes between TR and TL into the plant nucleus. Agrobacterium bearing each of these plasmids were used to co-infiltrate plants such as lettuce, resulting in the transient expression of hOAT.

FIG. 2 is a diagram of the bicistronic expression plasmid pSUNP4 which may be used to express both the heavy and light chain of hOAT from a single plasmid by transient expression in plant tissue.

FIG. 3 shows an elution profile of protein extracted from lettuce infiltrated with Agrobacterium containing the genes for hOAT heavy and light chains. The protein was applied to a Protein A column and eluted.

FIG. 4 shows an elution profile of protein applied to a Q Sepharose column isolated according to one aspect of the invention. The protein applied was that collected by elution from the ProteinA column used in FIG. 3.

FIG. 5A shows a Coomassie Blue-stained SDS-PAGE gel run under reducing conditions and hOAT protein fractions obtained according to one aspect of the invention. Lane 1, Molecular weight (Mr) standards; Lane 2, purified CHO produced hOAT; Lane 3, hOAT expressed in lettuce and eluted from a Protein A column. FIG. 5B shows a Western blot of SDS-PAGE-separated proteins isolated according to one aspect of the invention, probed with anti H+L antibody. Lane A, commercial IgG; Lane B, purified hOAT eluted from Protein A column; Lane C, negative control—extract from lettuce without agro-infiltration.

FIG. 6 shows the effect of various concentrations of sucrose for osmotic shock on the level of expression of hOAT in lettuce. The expression of hOAT expression is represented as mg antibody produced (ELISA based) per kilogram of lettuce material used for extraction.

### DETAILED DESCRIPTION OF THE INVENTION

The invention provides methods that make it possible to take advantage of protein production in grown, commercially available plants and provides a novel solution to the problem of procuring necessary amounts of heterologous proteins for use in biological assays in a short period of time. Methods of the invention provide biologically active heterologous proteins for use in assays that require at least about 50 μg-100 mg of protein, e.g., assays such as drug screening, protein characterization, binding assays, and animal testing.

In one aspect of the invention, the method comprises introducing an expression construct comprising a sequence encoding a heterologous protein or biologically active fragment thereof into a plant tissue and transiently expressing the protein in the plant tissue. The encoding sequence is operably linked to an expression control sequence capable of driving transcription of the encoding sequence in the cells and/or in the extracellular spaces of the plant tissue. Preferably, the expression construct comprises at least one T border sequence from T-DNA of a large tumor-inducing (“Ti”) plasmid. Also, preferably, the expression construct is comprised within a vector capable of replicating in at least the cells of an Agrobacterium species, such as Agrobacterium tumefaciens. In one aspect, the plant tissue comprises leaf tissue from an already grown plant (e.g., such as one obtainable from a store). Preferably, the plant comprises relatively large leaves (e.g., greater than about 3 inches in at least one dimension), e.g., the plant is lettuce (Lactuca sativa).

Definitions

The following definitions are provided for specific terms that are used in the following written description:

As used in the specification and claims, the singular form “a”, “an” and “the” include plural references unless the context clearly dictates otherwise. For example, the term “a cell” includes a plurality of cells, including mixtures thereof. The term “a protein” includes a plurality of proteins.

“Plant cells” as used herein includes plant cells or isolated or semi-isolated cells. “Plant tissue” includes differentiated and undifferentiated tissues of plants, including, but not limited to, roots, shoots, leaves, pollen, and seeds.

As used herein, “plant material” includes processed derivatives thereof, including, but not limited to: food products, food stuffs, food supplements, extracts, concentrates, pills, lozenges, chewable compositions, powders, formulas, syrups, candies, wafers, capsules and tablets.

As used herein, a “multi-subunit protein” is a protein containing more than one separate polypeptide or protein chain associated with each other to form a complex, where at least two of the separate polypeptides are encoded by different genes. In one preferred aspect, a multi-subunit protein comprises at least the immunologically active portion of an antibody and is thus capable of specifically combining with an antigen. For example, the multi-subunit protein can comprise the heavy and light chains of an antibody molecule or portions thereof. Multiple antigen combining portions can be encoded by different structural genes to generate multivalent antibodies.

In the case of a pharmaceutical product, the term “substantially pure” generally refers to a product of at least 90% pure, more preferably at least 95% and even more preferably at least 98% pure.

By “interstitial fluid” is meant the extract obtained from all of the area of a plant not encompassed by the plasmalemma, i.e., the cell surface membrane. The term is meant to include all of the fluid, materials, area or space of a plant that is not intracellular (wherein intracellular is defined as the contents contained within the cytoplasmic membrane) including molecules that may be released from the plasmalemma by this treatment without significant cell lysis. Synonyms for this term include, but are not limited to, “exoplasm”, “apoplasm”, “intercellular fluid”, “extracellular fluid” and “guttation fluid”.

The term “promoter” refers to the nucleotide sequence at the 5′ end of a gene that directs the initiation of transcription of the gene. Generally, promoter sequences are necessary, but not always sufficient, to drive the expression of a gene to which it is operably linked. In the construction of promoter/heterologous gene combinations, the gene is placed in sufficient proximity to and in a suitable orientation relative to a promoter such that the expression of the gene is controlled by the promoter sequence. The promoter is positioned preferentially upstream to the gene and at a distance from the transcription start site that approximates the distance between the promoter and the gene it controls in its natural setting. As is known in the art, some variation in this distance can be tolerated without loss of promoter function. As used herein, the term “operatively linked” means that a promoter is connected to a coding region in such a way that the transcription of that coding region is controlled and regulated by that promoter. Means for operatively linking a promoter to a coding region are well known in the art.

As used herein, an “expression control sequence” includes a promoter and may include, but is not limited to: one or more enhancer sequences, transcription termination sequences, polyadenylation sequences, 3′ or 5′ untranslated sequences, intronic sequences, ribosome binding sites, and other sequences that may stabilize or otherwise control expression of a gene in a plant cell. Expression control sequences may be endogenous (i.e., naturally found in a plant host) or exogenous (not naturally found in a plant host). Exogenous expression sequences may or may not be plant sequences so long as they are functional in a plant cell under selected conditions.

A “heterologous gene” or “heterologous coding sequence” is a gene that is exogenous to, or not naturally found in, the plant to be transformed or treated and that encodes a “heterologous polypeptide” or a biologically active fragment thereof. Heterologous gene sequences include viral, prokaryotic, and eukaryotic sequences. Prokaryotic encoding sequences include, but are not limited to, microbial sequences (e.g., for the production of antigens which may be administered as vaccines—viral sequences may also be used for this purpose). Eukaryotic coding sequences include mammalian sequences, but may also include sequences from non-mammals, even other plants, including but not limited to leader or secretion signal sequences, targeting sequences, and the like. In one preferred aspect, a heterologous gene nucleic acid encodes a human protein. The term “heterologous gene” or “heterologous coding sequence” includes, but is not limited to, naturally occurring, mutated, variant, chemically synthesized, genomic, cDNA, or any combination of such sequences. The reference to a “gene” encompasses full-length genes or fragments thereof encoding biologically active proteins.

As used herein, the term “a protein” is used to generically refer to the entire amino acid sequence encoded by a gene, to a processed or modified form thereof, or a biologically active fragment thereof (e.g., a polypeptide or peptide).

A “fusion protein” is a protein containing at least two different amino acid sequences linked in a polypeptide where the combination of sequences is not natively expressed as a single protein.

As used herein, a “T DNA border” refers to a DNA fragment comprising an about 25 nucleotide long sequence capable of being recognized by the virulence gene products of an Agrobacterium strain, such as an A. tumefaciens or A. rhizogenes strain, or a modified or mutated form thereof, and which is sufficient for transfer of a DNA sequence to which it is linked, to eukaryotic cells, preferably plant cells. This definition includes, but is not limited to, all naturally occurring T-DNA borders from wild-type Ti plasmids, as well as any functional derivative thereof, and includes chemically synthesized T-DNA borders. In one aspect, the encoding sequence and expression control sequence of an expression construct according to the invention is located between two T-DNA borders.

Plant Species

The early application of Agrobacterium infiltration for transient expression was based on poplar and Phaseolus (Kapila, et al., 1997), and then later extended to tobacco (Vaquero, et al., 1999) and both poplar and Phaseolus provide a suitable source of plant tissue for use in the instant invention. Other suitable plants include, but are not limited to: lettuce, alfalfa, mung bean, spinach, dandelion, radicchio, arugula, endive, escarole, chicory, artichoke, maize, potato, rice, soybean, Crucifera (e.g., Brassica, Arabidopsis) duckweed, maize, potato, rice, soybean, spinach, tomato and tobacco. Exemplary plants in the Brassica family include, but are not limited to: B. oleracea (e.g., cabbage, collards, cauliflower, broccoli, brussel sprouts, kale, kohlrabi); B. campestris (e.g., bok choy, pak choi, Chinese cabbage, celery cabbage, Siberian kale, turnip, mustard, rape, rutabaga, and radish); Brassica juncea (e.g., Brown and Indian Mustard); Brassica carinata (e.g., Abyssinian Mustard); Brassica napus (Rutabaga, Swede, Swede Turnip, Siberian Kale, Hanover Salad, canola); Brassica nigra (e.g., Black Mustard ); Rorippa nasturtium-aquatkum (e.g., Water Cress) and the like. A particularly preferred source of plant material is lettuce. Suitable lettuce plants include, but are not limited to: Butterhead, Crisphead, and Leaf lettuce (e.g., Oak leaf, Salad Bowl, frilly Red Leaf and crinkly Green Leaf). Additional types of lettuce are known in the art and described, for example, at http://www.thompson-morgan.coin/seeds/us/list_lettuce_{—}2.html.

Preferably, a suitable plant is commercially available year round and is able to support high-level transient expression of a reporter gene (e.g., such as GUS) operably linked to an expression control sequence. As used herein, “high level transient expression” refers to the capacity to express of at least about 250 μg, at least about 500 μg, at least about 750 μg, at least about 1 mg, at least about 2 mg, at least about 3 mg, at least about 4 mg, at least about 5 mg, at least about 10 mg, at least about 15 mg, at least about 25 mg, at least about 50 mg, at least about 75 mg, at least about 100 mg, at least about 150 mg, at least about 200 mg, or at least about 500 mg per kg of plant tissue mass. As used herein, “transient” refers to a period of time that is long enough to permit isolation of protein from a suitable plant tissue. Preferably, protein expression is at suitably high levels within at least about 1 day, at least about 2 days, at least about 3 days, at least about 4 days, at least about 5 days, after introduction of the expression construct into plant tissue. In one aspect, suitably high levels are obtained within 3-7 days and more preferably within 3-5 days, after introduction of an expression construct into the plant tissue.

Suitable plant tissue generally can be any part of the plant. In one preferred aspect, plant tissue is leaf tissue. In one aspect, the plant tissue is leaf tissue from a plant comprising leaves of at least about 3 inches in at least one dimension. However, leaf size is not limiting and in one aspect, the method is used to obtain transient protein expression in Arabidopsis.

In another aspect, a plant tissue is selected whose cells comprise little or no levels of proteases which digest heterologous proteins, e.g., less than about 5%, less than about 1%, less than about 0.1% of heterologous proteins expressed in the plant are digested during the period of time from introduction of nucleic acids expressing the heterologous protein to at least about the time when the protein is isolated from the plant tissue. Protease levels can be assayed for using methods routine in the art, including Western blot analysis of heterologous protein expression.

It is a particular advantage of the invention that already grown plants can be used as sources of plant tissues, including plants that have been harvested and stored for at least about a day, at least about 2 days, at least about 5 days, at least about 1 week, or at least about 2 weeks. Thus, plant tissue can be obtained from any general grocery store.

Lettuce provides a particularly suitable source of leaf tissue for use in methods according to the invention because lettuce is readily available and provides high levels of heterologous protein expression, stability and function. Additionally, lettuce cells comprise very low amounts of proteases recognizing heterologous proteins. Of the varieties of lettuce suitable for use, particularly preferred are those with red leaf phenotype. In addition to the high expression level of heterologous proteins observed in lettuce, an advantage of using a leafy lettuce is that the plant grows in a pattern that facilitates easy manipulation of the leaves without needing specifically designed equipment.

Corn is one of the most highly used crops for producing pharmaceutical proteins from stable transgenic plants. With corn, a heterologous protein is produced and stored in the seed. However, corn is difficult to transform, has a long generation time and is difficult to produce seed indoors. Corn is a crop that could benefit greatly from a transient expression system. It is now fairly common to use Agrobacterium for corn transformation, so that by treating corn embryos the transient expression system according to the invention can be used to accelerate the production of protein produced from maize in order to rapidly evaluate and confirm the utility/function of maize derived heterologous proteins.

Expression Cassettes

In preferred embodiments of the invention, wild type, mutant or modified varieties of lettuce (e.g., such as transgenic lettuce) are treated to express a gene of interest from a desired DNA construct. Such a construct minimally comprises a nucleic acid sequence encoding a desired protein operably linked to a promoter and/or other regulatory elements (i.e., an expression control sequence) to facilitate transcription of the gene and ultimately translation of the protein.

In one aspect, the expression construct is engineered to comprise the following, operably linked in the 5′ to 3′ direction: a promoter, gene and terminator. In another aspect, the gene construct comprises multiple coding regions operably linked on a common plasmid or co-transformed into the plants (such co-transformed constructs are collectively encompassed by the term “gene construct” as used herein). Multiple genes may be encoded as separate cistrons or as part of polycistronic units. In a further aspect, the gene construct comprises one or more IRES elements.

It is not necessary for the gene construct to contain a selectable marker nor is it required that the DNA construct be devoid of “tumor inducing” genes as would be required for production of morphologically normal stable transgenic plants.

Proteins

There is no preconceived limitation as to the proteins to be produced by this invention, but there are certain categories of proteins that may be of particular relevance, given the need to produce certain products under regulated and reproducible conditions. In particular, this would include all classes of pharmaceutical and/or diagnostic proteins for which Good Manufacturing Practices and validated methods must be used during the course of production.

Proteins also may be expressed for their utility as nutraceuticals and cosmeceuticals, since these products are used for direct ingestion, injection or application (e.g., topical administration) to humans. Protein also may be expressed which are useful in the production of similarly regulated veterinarian products.

Exemplary proteins which may be produced, include, but are not limited to: growth factors (e.g., such as Platelet-Derived Growth Factor, Insulin-like Growth Factor, etc.), receptors, ligands, signaling molecules; kinases, enzymes, hormones, tumor suppressors, blood clotting proteins, cell cycle proteins, telomerases, metabolic proteins, neuronal proteins, cardiac proteins, proteins deficient in specific disease states, antibodies, T-cell receptors (TCR), Major Histocompatibility Complexes (MHC), antigens, proteins that provide resistance to diseases, antimicrobial proteins, interferons, and cytokines.

In one aspect, antigen encoding sequences including sequences for inducing protective immune responses (e.g., as in a vaccine formulation). Such suitable antigens include but are not limited to microbial antigens (including viral antigens, bacterial antigens, fungal antigens, parasite antigens, and the like); antigens from multicellular organisms (such as multicellular parasites); allergens; and antigens associated with human or animal pathologies (e.g., such as cancer, autoimmune diseases, and the like). In one preferred aspect, viral antigens include, but are not limited to: HIV antigens; antigens for conferring protective immune responses to smallpox (e.g., vaccinia virus antigens); anthrax antigens; rabies antigens; and the like. Vaccine antigens can be encoded as multivalent peptides or polypeptides, e.g., comprising different or the same antigenic encoding sequences repeated in an expression construct, and optionally separated by one or more linker sequences.

Plants also may be used to express one or more genes to reproduce enzymatic pathways for chemical synthesis or for industrial processes.

In one aspect, nucleic acid sequences are chosen encoding desired proteins wherein the nucleic acid sequences are designed to provide codons preferred by lettuce or the plant that might eventually be used for large-scale production of the desired protein if that codon selection does not reduce expression in the transient system below useful levels. The characteristics of codon usage for several plants are available and are described in Wada et al., “Codon Usage Tabulated From The GenBank Genetic Sequence Data,” Nucleic Acids Research 19 (Supp.): 1981-1986 (1991), for example.

As described further below, in one aspect, the invention provides a method for expressing a plurality of recombinant proteins. Such proteins may be expressed upon co-infiltration of independent constructs or may be expressed from polycistronic expression units described further below. Such proteins can include those that in their native state require the coordinate expression of a plurality of structural genes in order to become biologically active. In one aspect, the protein requires the assembly of a plurality of subunits to become active. In another aspect, the protein is produced in immature form and requires processing, e.g., proteolytic cleavage, or modification (e.g., phosphorylation, glycosylation, ribosylation, acetylation, farnesylation, and the like) by one or more additional proteins to become active.

Non-limiting examples of such proteins include heterodimeric or heteromultimeric proteins, such as T Cell Receptors, MHC molecules, other proteins of the immunoglobulin superfamily (including fragments and single chain variants), nucleic acid binding proteins (e.g., replication factors, transcription factors, etc.), enzymes, abzymes, receptors (particularly soluble receptors), growth factors, cell membrane proteins, differentiation factors, hemoglobin like proteins, multimeric kinases, and the like.

In preferred aspects of the invention, expression cassettes encode human proteins (i.e., proteins expressed in humans) or encode proteins comprising human polypeptide regions comprised within otherwise non-human proteins.

In one particularly preferred aspect, the expression cassette encodes one or more genes for monoclonal antibodies. Such genes can be obtained from murine, human and/or other animal sources. Alternatively, they can be synthetic, e.g., chimeric or modified forms of the genes encoding the heavy chain or light chain components of an antibody molecule. The order of the coding regions on the construct, e.g., heavy then light, or light then heavy, is not important. Genes coding for heavy and light chain polypeptides (e.g., such as variable heavy and variable light domain polypeptides) can be derived from cells producing IgA, IgD, IgE, IgG or IgM. Methods for preparing fragments of genomic DNA from which immunoglobulin variable region genes can be cloned are well known in the art. See, for example, Herrmann et al., Methods in Enzymol. 152: 180-183 (1987); Frischauf, Methods in Enzymol. 152:183-190 (1987); Frischauf, Methods in Enzymol. 152: 199-212 (1987). In one preferred embodiment, such as described below, such genes are encoded as part of polycistronic units.

Regulatory Elements

Suitable regulatory elements for generating a particular construct will be selected based on the type of recombinant protein to be expressed. In general, the ability to express at high levels in the infiltrated plant tissue is desired.

Plant Promoters

The gene constructs used may include all of the genetic material of the gene or portions thereof which encode biologically active protein fragments. Preferably encoding sequences are operably linked to expression control sequences. Expression control regions include and such sequences as promoters, enhances, IRES elements, etc. Expression control sequences can either require some external stimuli to induce expression, such as the addition of a particular nutrient or agent, change in temperature, etc., or can be designed to express an encoded protein immediately and/or spontaneously during infiltration and/or incubation of the plant tissue.

Thus, constitutive or regulated promoters may control the expression of a gene encoding a desired protein. Regulated promoters may be environmentally signaled, or controllable by means of chemical inducers or repressors and such agents may be of natural or synthetic origin and the promoters may be of natural origin or engineered. Promoters also can be chimeric, i.e., derived using sequence elements from two or more different natural or synthetic promoters.

Preferably, a promoter used in the construct yields a high expression level of the gene, allowing for accumulation of the protein to be at least about at least about 250 μg, at least about 500 μg, at least about 750 μg, at least about 1 mg, at least about 2 mg, at least about 3 mg, at least about 4 mg, at least about 5 mg, at least about 10 mg, at least about 15 mg, at least about 25 mg, at least about 50 mg, at least about 75 mg, at least about 100 mg, at least about 150 mg, at least about 200 mg, or at least about 500 mg per kg of plant tissue mass (e.g., leaf tissue biomass).

In the present invention, the Arabidopsis Actin 2 promoter, the OCS3(MAS) promoter, the CaMV 35S promoter, and figwort mosaic virus 34S promoter are preferred. However, other constitutive and inducible promoters can be used. For example, the ubiquitin promoter has been cloned from several species for use in plants (e.g., sunflower (Binet et al., Plant Science 79: 87-94 (1991); and maize (Christensen et al., Plant Molec. Biol. 12:619-632 (1989)). Further useful promoters are the U2 and U5 snRNA promoters from maize (Brown et al., Nucleic Acids Res. 17: 8991 (1989)) and the promoter from alcohol dehydrogenase (Dennis et al., Nucleic Acids Res. 12: 3983 (1984)).

In another aspect, a regulated promoter is operably linked to the gene. Regulated promoters include, but are not limited to, promoters regulated by external influences (such as by application of an external agent, e.g., such as chemical, light, temperature, and the like), or promoters regulated by internal cues, such as regulated developmental changes in the plant. Regulated promoters are useful to induce high-level expression of a desired gene specifically at, or near, the time of harvest. This may be particularly useful in cases where the desired protein limits or otherwise constrains growth of the plant, or is in some manner, unstable. Such promoters may be desirable when the expression construct is expected to be used in the production of transgenic plants as well as in transient expression assays.

Plant promoters that control the expression of transgenes in different plant tissues are known to those skilled in the art (Gasser & Fraley, Science 244:1293-99 (1989)). The cauliflower mosaic virus 35S promoter (CaMV) and enhanced derivatives of CaMV promoter (Odell et al., Nature, 3(13):810 (1985)), actin promoter (McElroy et al., Plant Cell 2:163-71 (1990)), AdhI promoter (Fromm et al., Bio/Technology 8:833-39 (1990), Kyozuka et al., Mol. Gen. Genet. 228:40-48 (1991)), ubiquitin promoters, the Figwort mosaic virus promoter, mannopine synthase promoter, nopaline synthase promoter and octopine synthase promoter and derivatives thereof are considered constitutive promoters. Regulated promoters are described as light inducible (e.g., small subunit of ribulose biphosphatecarboxylase promoters), heat shock promoters, nitrate and other chemically inducible promoters (see, for example, U.S. Pat. Nos. 5,364,780; 5,364,780; and 5,777,200).

Tissue specific promoters are used when there is reason to express a protein in a particular part of the plant. Leaf specific promoters may include the C4PPDK promoter preceded by the 35S enhancer (Sheen, EMBO, 12:3497-505 (1993)) or any other promoter that is specific for expression in the leaf.

Generally, any plant expressible genetic construct is suitable for use in the methods of the invention. Particular promoters may be selected in consideration of the type of recombinant protein being expressed.

Of particular interest for this invention is the use of a synthetic promoter derived from multiple units of the OCS enhancer and the core promoter from MAS wherein the OCS and MAS elements are from Agrobacterium (Gelvin et al., U.S. Pat. No. 5,955,646). This promoter has been shown to be particularly strong following infiltration and treatment with the plant hormone 2,4-D, which is also used as an herbicide at higher concentrations.

Targeting Sequences

In preferred embodiments, expression products are targeted to a specific location in a plant cell, such as the cell membrane, extracellular space or a cell organelle, e.g., a plastid, such as a chloroplast. In a preferred embodiment, expression products are targeted to the extracellular space, thus enabling purification based on the isolation of the intracellular fluids. See, for example, U.S. Pat. No. 6,096,546, U.S. Pat. No. 6,284,875, and WO 0,009,725.

Proteins can be targeted to specific sub-cellular or extracellular locations by virtue of targeting sequences. In some cases the sequence of amino acids is synthesized as the amino terminal portion of the polypeptide and is cleaved by proteases, after, or during, the translocation or localization process. For instance, the model of the protein secretion pathway in eukaryotes is that following ribosome binding to mRNA and initiation of translation the nascent polypeptide chain emerges. If it is a protein destined for secretion, the emerging amino terminus of the protein is recognized by signal recognition particle (SRP) that brings about a temporary stalling of translation while an mRNA, ribosome and SRP complex docks with the endoplasmic reticulum (ER). After docking, translation resumes, although now the polypeptide chain is co-translationally translocated through to the ER lumen.

The signal sequences for targeting proteins to the endomembrane system for localization in the vacuole or for secretion are similar in plants and animals. Signaling peptides may be adapted for use in the present invention (e.g., prepared with suitable ends for cloning in-frame with any other gene) in accordance with standard techniques.

In one aspect, an expression cassette encoding a desired protein comprises a signal sequence fused in frame to sequences encoding the desired protein. In one preferred aspect, the signal sequence is one that can direct the expression product of the gene to a secretory pathway.

As antibodies are normally secreted proteins, the secretion process plays an important role in the production of the mature antibody molecules. To accomplish this in plants, the genes are synthesized or otherwise obtained (e.g., cloned) having either their native mammalian signal peptide encoding region, or as a fusion in which a plant secretion signal peptide is substituted for the signal peptide of and operably linked to the gene of interest. The fusion between the signal peptide and the protein should be such that upon processing by the plant, the resultant amino terminus of the protein is identical to that which is generated in the human host. However targeting to the chloroplast is also anticipated.

In a preferred embodiment, the signal sequence from calreticulin (Borisjuk et al., Nature Biotechnology 17: 466-69 (1999)) is used. A more preferred embodiment uses the subtilase sequence from tomato (Janzik et al., 2000). It has been demonstrated that these plant signal peptides are efficient at targeting foreign proteins to the apoplastic space of the plant (see, e.g., Janzik et al., 2000). Other plant protein signal peptides may also be used such as those described for barley (α-amylase, During et al. Plant Molecular Biology 15: 287-93 (1990); Schillberg et al. Transgenic Research 8: 255-63 (1999)).

Targeting proteins to the endomembrane system of a plant is a preferred embodiment of the present invention for those proteins that normally require amino-terminal processing to achieve their mature form, because it provides for the proper maturation of the amino terminus of the protein. Further, localization to specific regions of the endomembrane system can be accomplished if the protein of interest either has, or is, engineered to contain additional targeting information (see, e.g., as described in: Voss et al., Mol. Breeding 1: 39-50 (1995); During et al., Plant Mol. Biol. 15: 281-93 (1990); Baum et al., Mol. Plant-Microbe Interact. 9: 382-87 (1996); DeWilde et al., Plant Sci. 114: 231-41 (1996); Ma et al., Immunology 24: 131-38 (1994); Schouten et al., Plant Mol. Biol. 30: 781-93 (1996); Firek et al., Plant Mol. Biol. 23: 861-70 (1993); Artsaenko et al., Plant J. 8: 745-50 (1995); Conrad & Fiedler, Plant Mol. Biol. 38: 101-09 (1998)).

Targeting to organelles such as plastids (e.g., chloroplast and mitochondria) is also advantageous for achieving the desired amino-terminal maturation because targeting to either of these locations is dictated by an amino-terminal signal sequence that subsequently undergoes a cleavage event. In preferred embodiments, the signaling peptides direct the expression products to a plastid (e.g., a chloroplast) or other subcellular organelle. An example is the transit peptide of the small subunit of the alfalfa ribulose-biphosphate carboxylase (Khoudi et al., Gene 197: 343-5 (1997)). A peroxisomal targeting sequence refers to any peptide sequence, either N-terminal, internal, or C-terminal, that can target a protein to the peroxisomes, such as the plant C-terminal targeting tripeptide SKL (Banjoko et al., Plant Physiol. 107: 1201-08 (1995)).

Additionally, or as an alternative to targeting proteins to specific subcellular locations, in one aspect, “epitope tags” and/or site-specific cleavage sites are added to create a fusion protein. The utility of such tags is that they can provide a convenient purification mechanism. For instance, a small peptide comprising the critical amino acid sequence from biotin for binding to streptavidin can be engineered on to the 5′ end of a gene of interest. The newly synthesized protein can then be captured by many known methods fundamentally based on biotin:straptavidin binding. If it is desirable to remove the “biotin-like” peptide from the protein, it is possible to also include a protease recognition site. The protease recognition site can be inserted downstream from the “epitope tag” sequence and just before the sequence encoding the mature form of the desired protein. Those skilled in the art will recognize that there are numerous choices for epitope tags and proteases (such as factor Xa, Tobacco Etch Virus protease, enterokinase, etc.) and that the choice of the preferred site and protease may depend on the specific protein amino acid and DNA sequence in question.

As described above, the selection of regulatory elements, such as promoters, enhancers, IRES elements, and signal sequences will generally depend on the type of protein being expressed. For example, in one aspect, some preferred constructs for the purpose of making an IgG would include constructs having 5′ OCS3MAS promoter: subtilase (any organism) signal peptide: coding region for the mature portion of the IgG heavy chain gene: translational stop signals: transcriptional stop and polyadenylation sequence, as well as a second construct containing similar elements as above, replacing the heavy chain gene with the light chain gene (i.e. two vectors, referred to herein as “binary” or “dual” vectors). Alternatively, in another preferred embodiment, the heavy chain and light chain genes are on the same DNA construct. In yet another embodiment, the heavy chain and light chain genes are expressed from the same promoter on the same DNA construct separated by an IRES element.

Other Sequences

The expression construct may be part of an expression vector and can include additional desirable sequences such as bacterial origins of replication (Agrobacterium and/or E. coli origins of replication), reporter genes that function in bacteria such as Agrobacterium and/or plant cells (e.g., GUS, GFP, EGFP, BFP, β-galactosidase and modified forms thereof) and selectable marker genes (e.g., antibiotic resistance genes, and the like). To this end, the foreign DNA used in the method of this invention may also comprise a marker gene, the expression of which allows the separation of transformed cells from non-transformed cells during initial cloning stages. Such a marker gene generally encodes a protein which allows one to phenotypically distinguish transformed cells from untransformed cells. In plants, the selectable marker gene may thus also encode a protein that confers resistance to a herbicide, such as a herbicide comprising a glutamine synthetase inhibitor, such as phosphinothricin (see, e.g., EP 0 242 236; EP 0 242 246; De Block et al., 1987, EMBO J. 6: 2513-2518). However, it is an advantage of the transient protein production methods according to the invention that marker genes are not required to isolate heterologous proteins from plant tissues into which expression constructs/vectors are introduced.

Additional sequences that can be fused to sequences encoding heterologous proteins include, but are not limited to: coiled-coil sequences (e.g., as described in Martin et al., EMBO J 13(22): 5303-5309 (1994)); minibody sequences or sequences composed of a minimal antibody complementarity region (see, e.g., Bianchi et al., J. Mol. Biol. 236(2): 649-59 (1994)); stabilizing sequences, dimerization sequences, linker sequences, myristilation sequences (see, e.g., as described in U.S. patent Publication No. 2002/0146710), Fc regions (e.g., for producing immunoadhesins) and the like.

Libraries of Expression Constructs

In one aspect, a plurality of expression constructs are generated comprising substantially identical coding sequences (e.g., greater than about 50% identical, greater than about 75% identical, greater than about 90, 95%, or 99% identical) for expression and testing of variant protein sequences in transient protein expression systems according to the invention.

In one aspect, the encoding portions of the constructs are randomized. Constructs can be fully randomized or biased in their randomization (i.e., randomized or at one or more selected positions). In one aspect, a library of expression constructs is generated which comprises a sufficiently diverse population such that at least one protein encoded by an expression construct in the plurality of constructs has a desired biological activity (e.g., such as the ability to bind to a particular binding partner such as an antigen). In another aspect, the plurality of expression constructs comprises greater than about 100, greater than about 500, greater than about 1×10³, greater than about 1×10⁴, greater than about 1×10⁵, greater than about 1×10⁶, greater than about 1×10⁷, greater than about 1×10⁸, or greater than about 1×10⁹ variant encoding sequences. Preferably, the diversity of the library is such that it comprises about 1×10⁷-1×10⁹ different variant encoding sequences. One or more amino acids of a protein may be randomized at a time. In one aspect, about one, about two, about three, about four, about 5, or about 6 or more amino acids are randomized at a time. In one aspect, for a protein comprising “n” amino acids, each one of the n amino acids is independently randomized and the protein is tested for activity. Randomization may be biased in that particular region(s) of a heterologous protein may be varied (such as an antigen binding site or a particular amino acid) while other regions remain constant.

Methods of mutating nucleic acid sequences for the directed evolution of proteins are described in Leung et al. Technique 1: 11-15 (1989); Cadwell and Joyce, PCR Methods Appl. 2: 28-33 (1992); Shafikhani et al., Biotechniques 23: 304-310 (1997); Wan et al., Proc. Natl. Acad. Sci. U.S.A. 95: 12825-12831 (1998); You and Arnold, Protein Eng. 9: 77-83 (1996); Cherry et al. Nat. Biotechnol. 17: 379-384 (1999); Tukey et al., J Immunol Methods 270(2): 247-57 (2002); Cho et al., Mol. Biol. 297(2): 309-19 (2000), for example.

Plant tissue samples can be infiltrated with the plurality of expression constructs as described further below and tissues/cells which express proteins having desired types and/or levels of biological activity can be selected for in high throughput assays. An expression construct can be rescued from one or more cells in a sample showing a desired type/level of activity and sequenced or otherwise characterized to identify the variant sequence associated with the type/level of activity. The construct can be reintroduced into one or more additional plant tissue samples to confirm the result, either before or after the sequence of the construct is characterized. A second round of biased randomization may be used to change unaltered sites of the protein and/or selected regions of the protein to identify proteins with enhanced properties (e.g., proteins which have enhanced binding affinity for a particular binding partner).

In one aspect, the method is used to mimic the natural selection process involved in the generation of an antibody, i.e., identifying expression constructs which bind to a particular antigen, then mutating the constructs and performing a second round of selection to identify constructs which provide the highest affinity for the particular antigen.

Agrobacterium Transformation and Culture Preparation

Transformation of plants with Agrobacterium and its use in generation of stable plant transgenics has been well documented. The interaction of an Agrobacterium cell comprising a T- DNA border sequence with a plant cell results in the transfer of a single strand copy of Agrobacterium T-DNA complexed with proteins to the plant nucleus. For stable transformation, the T-DNA is integrated into the nuclear DNA.

Although the process is apparently quite efficient, the non-integrated copies of T-DNA are able to be transiently transcribed resulting in the short-term expression of the T-DNA genes and any other genes that are co-transformed. Since the transient expression is not dependent on integration of DNA or regeneration of plants, it is possible to use the more virulent strains of Agrobacterium without the need to use disarmed vectors (i.e., vectors which no longer contain tumor producing genes), although the latter may also be used.

Suitable disarmed vectors include the SEV series in which the right border of the T-DNA, together with the phytohormone genes coding for cytokinin and auxin, are removed and replaced by a bacterial kanamycin resistance gene while the left border and a portion of the Left Inside Homology (LIH) sequences are left intact, and the pGV series, in which the phytohormone genes are excised and substituted by part of pBR322 vector sequence and the left and right border sequences as well as the nopaline synthase gene of the Ti plasmid are conserved. Intermediate vectors may be used in combination with helper sequences. In some preferred aspects, binary vectors are used, comprising a T-region in one vector, and a vir region in another vector. Binary vectors are known in the art and described in U.S. Pat. No. 4,940,838, EP 120516 B1, and U.S. Pat. No. 5,464,763, for example.

Suitable strains of Agrobacterium include wild type strains (e.g., such as Agrobacterium tumefaciens) or strains in which one or more genes is mutated to increase transformation efficiency, e.g., such as Agrobacterium strains wherein the vir gene expression and/or induction thereof is altered due to the presence of mutant or chimeric virA or virG genes (e.g. Chen and Winans, 1991, J. Bacteriol. 173: 1139-1144; and Scheeren-Groot et al., 1994, J. Bacteriol. 176:6418-6246). In another embodiment, Agrobacterium strains comprising an extra virG gene copies, such as the super virG gene derived from pTiBo542, preferably linked to a multiple-copy plasmid, as described in U.S. Pat. No. 6,483,013, for example.

Other suitable strains include, but are not limited to: A. tumefaciens C58C1 (Van Larebeke et al., Nature 252: 169-170 (1974)), A136 (Watson et al., J. Bacteriol 123: 255-264 (1975)); LBA401 1 (Klapwijk et al., J. Bacteriol 141: 128-136 (1980)), LBA4404 (Hoekema et al., Nature 303: 179-180 (1983)); EHA101 (Hood et al., J. Bac. 168: 1291-1301 (1986)); EHA105 (Hood et al., Trans Res. 2: 208-218 (1993)); AGL1 (Lazo et al., Bio/Technology 2: 963-967 (1991)); A281 (Hood et al., supra (1986)).

In one aspect, the invention provides a simplified method of processing Agrobacterium. Preferably, a strain of Agrobacterium is cultured to an Optical Density (O.D.) at 600 nm of 2.5-3.5 in a suitable culture medium. The cells are generally diluted to an O.D. of 2.5. The Agrobacterium cells are then directly contacted (i.e., without an initial concentration step or centrifugation step) with a plant tissue, using approximately 1-3 volumes of a suspension of Agrobacterium in culture medium per volume of plant tissue, preferably about 2-3 volumes. In one embodiment, less than about 4 L of bacterial culture per 1 kg of plant material provides at least about 250 μg, at least about 500 μg, at least about 750 μg, at least about 1 mg, at least about 2 mg, at least about 3 mg, at least about 4 mg, at least about 5 mg, at least about 10 mg, at least about 15 mg, at least about 25 mg, at least about 50 mg, at least about 75 mg, at least about 100 mg, at least about 150 mg, at least about 200 mg, or at least about 500 mg of heterologous protein.

The method can thus be performed in fewer steps, requiring less manpower and 15-20 fold reductions in the cost of production compared to other methods used.

Vacuum Infiltration

In one particularly preferred embodiment, a surfactant is added to the Agrobacterium suspension to enhance the yield of heterologous protein from the plant tissue. In one aspect, Agrobacterium cells or portions thereof are infiltrated into the host plant tissue for expression of the expression construct/expression vector. Preferably, this step is performed in the presence of a vacuum.

As used herein, the term “surfactant” refers to a surface-active agent that generally comprises a hydrophobic portion and a hydrophilic portion (see, e.g., Bhairi, A Guide to the Properties and Uses of Detergents in Biological Systems, Calbiochem-Novabiochem Corp. 1997). Surfactants may be categorized as anionic, nonionic, zwitterionic, or cationic, depending on whether they comprise one or more charged groups. Anionic surfactants contain a negatively charged group and have a net negative charge. Nonionic surfactants contain non-charged polar groups and have no charge. These surfactants are generally the reaction products of alkylene oxide with alkyl phenol, or primary or secondary alcohols, or are amine oxides, phosphine oxides or dialkyl sulphoxides.

Exemplary nonionic surfactants include, but are not limited to: t-octylphenoxypolyethoxyethanol (Triton X-100), polyoxyethylenesorbitan monolaurate (Tween 20), polyoxyethylenesorbitan monolaurate (Tween 21), polyoxyethylenesorbitan monopalmitate (Tween 40), polyoxyethylenesorbitan monostearate (Tween 60), polyoxyethylenesorbitan monooleate (Tween 80), polyoxyethylenesorbitan monotrioleate (Tween 85), (octylphenoxy)polyethoxyethanol (IGEPAL CA-630/NP-40), triethyleneglycol monolauryl ether (Brij 30), and sorbitan monolaurate (Span 20).

A zwitterionic surfactant contains both a positively charged group and a negatively charged group, and has no net charge. Suitable zwitterionic surfactants include, but are not limited to: betaines, such as carboxybetaines, sulfobetaines (also known as sultaines), amidobetaines and sulfoamidobetaines, such amay comprise C₈-C₁₈, preferably C₁₀-C₁₈, alkyl betaines, sulfobetaines, amidobetaines, and sulfoamidobetaines, for example, laurylamidopropylbetaine (LAB) type-betaines, n-alkyldimethylammonio methane carboxylate (DAMC), n-alkyldimethylammonio ethane carboxylate (DAEC) and n-alkyldimethylammonio propane carboxylate (DAPC), n-alkylsultaines, n-alkyl dimethylammonio alkyl sulfonates, N-alkyl dimethylammonio ethane sulfonate (DAES), n-alkyl dimethylammonio propane sulfonate (DAPS) and n-alkyl dimethylammonio butane sulfonate (DABS), hexadecyl dimethylammonio propane sulfonate, n-alkylamidomethane dimethylammonio methane carboxylates, n-alkylamido methane dimethylammonio ethane carboxylate, laurylamidopropylbetaine (LAB), n-alkylamidomethane dimethylammonio methane sulfonate, n-alkylamidoethane dimethylammonio ethane sulfonate and n-alkylamidopropane dimethylammonio propane sulfonate, 3-[(3-cholamidopropyl)dimethylammonio]-1-propanesulfonate (CHAPS), and 3-[(3-cholamidopropyl)dimethylammonio]-2-hydroxy-1-propanesulfonate (CHAPSO), phospholipids (e.g., phosphatidylethanolamines, phosphatidylglycerols, phosphatidylinositols, diacyl phosphatidyl-cholines, di-O-alkyl phosphatidylcholines, lysophosphatidylcholines, lysophosphatidylethanolamines, lysophosphatidylglycerols, lysophosphatidylinositols, saturated and unsaturated fatty acid derivatives (e.g., ethyl esters, propyl esters, cholesteryl esters, coenzyme A esters, nitrophenyl esters, naphtyl esters, monoglycerids, diglycerids, and triglycerides, fatty alcohols, fatty alcohol acetates, and the like), lipopolysaccharides, glyco- and shpingolipids (e.g., ceramides, cerebrosides, galactosyldiglycerids, gangliosides, lactocerebrosides, lysosulfatides, and the like).

A “cationic surfactant” has a positively charged group under the conditions of infiltration. Suitable cationic surfactants include, but are not limited to: quaternary amines or tertiary amines. Exemplary quaternary amine surfactants include, but are not limited to, cetylpyridinium chloride, cetyltrimethylammonium bromide (CTAB; Calbiochem # B22633 or Aldrich #85582-0), cetyltrimethylammonium chloride (CTACl; Aldrich #29273-7), dodecyltrimethylammonium bromide (DTAB, Sigma #D-8638), dodecyltrimethylammonium chloride (DTACI), octyl trimethyl ammonium bromide, tetradecyltrimethylammonium bromide (TTAB), tetradecyltrimethylammonium chloride (TTACI), dodecylethyidimethylammonium bromide (DEDTAB), decyltrimethylammonium bromide (DlOTAB), dodecyltriphenylphosphonium bromide (DTPB), octadecylyl trimethyl ammonium bromide, stearoalkonium chloride, olealkonium chloride, cetrimonium chloride, alkyl trimethyl ammonium methosulfate, palmitamidopropyl trimethyl chloride, quaternium 84 (Mackernium NLE; Mcintyre Group, Ltd.), and wheat lipid epoxide (Mackernium WLE; Mcintyre Group, Ltd.). Exemplary ternary amine surfactants include, but are not limited to, octyldimethylamine, decyidimethylamine, dodecyidimethylamine, tetradecyldimethylamine, hexadecyidimethylamine, octyldecyldimethylamine, octyidecylmethylamine, didecylmethylamine, dodecylmethylamine, triacetylammonium chloride, cetrimonium chloride, and alkyl dimethyl benzyl ammonium chloride. Additional classes of cationic surfactants include, but are not limited to: phosphonium, imidzoline, and ethylated amine groups.

Anionic surfactants are generally water-soluble alkali metal salts of organic sulfates and sulfonates. These include, but are not limited to: potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.).

Co-surfactants such as a short-chain alcohol such as ethanol, 1-propanol, and 1-butanol, may additionally be used.

Combinations of any of the above surfactants may be used. Surfactants not specifically listed above are further encompassed within the scope of the invention.

Amounts of surfactants used will vary with the type of surfactant and plant tissue being treated (i.e., the thickness of the wax covered surface of a leaf, etc.). Generally, surfactants are used in concentrations ranging from 0.005% to about 1% of the volume of the Agrobacterium suspension. Preferably, concentrations range from 0.005% to about 0.5%, and more preferably, from about 0.005% to about 0.05%. Generally, lower levels of ionic surfactants will be used than nonionic surfactants.

In one preferred aspect, a nonionic surfactant, such as Tween 20 is used.

In addition to incorporating a surfactant, adding an osmotic shock step to augment the vacuum shock can be used increase protein expression. Therefore, in one aspect, an osmotic shock agent such as sucrose is used to increase protein expression. Suitable concentrations of the osmotic shock agent range from 20 g/L to 100 g/L. In one aspect, where the plant tissue is lettuce, about 60 g/L of sucrose is used.

In an exemplary method, recombinant Agrobacterium cultures (i.e., comprising expression constructs according to the invention) are grown for approximately two days in modified YEB medium (yeast extract 6 g/L, peptone 5 g/L, magnesium sulfate 2 mM, and sucrose 5 g/L) supplemented with appropriate antibiotics to select for resistance determinants found on the vectors and the host. To grow cells for transient expression, the starter Agrobacterium cultures are diluted 1:50 into fresh modified YEB medium. Antibiotics, 50 mM potassium phosphate buffer (pH 5.8) and 20 μM acetosyringone are added. After 18-24 hours incubation at 28° C., cells reach an absorbance (also referred to as Optical Density or O.D.) at 600 nm of 2.5-3.5. The cells are preferably diluted to an absorbance at 600 nm of 2.5, if necessary, using the same medium.

The cells are then supplemented with a sucrose and acetosyringone to give a maximum of 220 μM acetosyringone and 60 g/L of sucrose. The suspension is incubated for about 1 hour at 22° C. and then used for infiltration.

The cells are then infiltrated directly under vacuum without any centrifugation or concentration step, eliminating the need for a resuspension step. This modification permits direct vacuum infiltration with freshly grown Agrobacterium suspension, eliminating the need to centrifuge the cells from a logarithmic phase culture and to resuspend them in Murishigi Skoog (MS) medium (Kapila et al., supra (1997)). The growth of Agrobacterium cells to an O.D._{600 nm} of 2.5-3.5 instead of 0.7-0.8 O.D._{600 nm} (Kapila et al., 1997), use of modified YEB medium and potassium phosphate buffer instead of MES, did not alter expression levels or infiltration efficiency but significantly reduced the cost and effort required for this part of the process.

In one aspect, e.g., where the plant tissue is from lettuce, the plant material is immersed into a pre-incubated Agrobacterium suspension together with 100 μg/ml of 2,4-D and 0.005% of Tween 20, in a beaker. The beaker is placed in a vacuum desiccator, and a vacuum (equivalent to a 29″ column of water or about 7 kPa) is applied for 20 minutes followed by vacuum shock resulting from the quick release of the vacuum. The use of a whole head of lettuce is significantly more convenient compared to a mass of unorganized leaves, as described previously (Kapila et al., supra (1997); Vaquero et al., supra (1999)). In general, whole heads of lettuce appear to give better expression levels compared to equivalent amounts of leaf biomass. The method can be readily scaled up by the simultaneous treatment of numerous lettuce heads or larger quantities of leaves. One Agrobacterium cell suspension can be used at least twice for vacuum-infiltration without significant reduction of efficiency of expression, which further reduces the cost of production and labor.

In one aspect, a whole head of lettuce, approximately, 300-400 g is used. In another aspect, separate leaves are used for pilot experiments to optimize amounts and combinations of surfactants and/or osmotic agents.

Preferably, following vacuum-infiltration the heads of lettuce are incubated in light for about 3-7 days at room temperature, then homogenized for protein extraction. Monoclonal antibodies and other pharmaceutically important proteins are purified from plant homogenates using appropriate purification procedures.

The process is simple, comprises fewer steps and results in a dramatic increase in expression of heterologous proteins compared to other prior art methods.

Isolation of Proteins

After harvesting, protein isolation may be performed using methods routine in the art. For example, at least a portion of the biomass may be homogenized, and recombinant protein extracted and further purified. Extraction may comprise soaking or immersing the homogenate in a suitable solvent. Proteins may also be isolated from interstitial fluids of plants, for example, by vacuum infiltration methods, as described in U.S. Pat. No. 6,284,875.

Purification methods include, but are not limited to, immunoaffinity purification and purification procedures based on the specific size of a protein or protein complex, electrophoretic mobility, biological activity, and/or net charge of the heterologous protein to be isolated, or based on the presence of a tag molecule in the protein.

Characterization of the isolated protein can be conducted by immunoassay or by other methods known in the art. For example, proteins can analyzed on SDS-PAGE gels by Western blotting, or by Coomassie blue staining when the protein is substantially purified. The isolated proteins can be used to assay biological activity, characterize protein structure (e.g., in crystallization assays), perform efficacy testing in non-animal human models of disease, screen for optimal protein activity and/or optimal pharmaceutical characteristics, and the like.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated as being incorporated by reference herein.

### EXAMPLES

The present invention will now be described by way of several working examples. These examples are for purposes of illustration and are not meant to limit the invention in any way.

### Example 1

### Production of hOAT by a Novel Rapid Protein Expression System

The methods described herein provide significant changes from the transient expression systems such as taught by Kapila, et al., supra (1977), and result in dramatically improved expression of high quality product at low cost and with fewer steps.

TABLE 1
Comparison of Transient Expression

| Step | Kapila Method | Method of the Invention |
|---|---|---|
| 1 | Grow Agrobacterium in | Grow Agrobacterium in |
| | YEB (see text). | modified YEB (see text). |
| 2 | Pre-infiltration growth of | Pre-infiltration growth of |
| | Agrobacterium | Agrobacterium in |
| | in YEB + MES | YEB + PO₄ (pH |
| | (pH 5.6) + 20 μM | 5.6) + 20 μM acetosyringone. |
| | acetosyringone. Dilute 1:500 | Dilute 1:50 and grow overnight to |
| | and grow overnight | O.D.₆₀₀ₙₘ˜2.5-3.5. |
| | to O.D.₆₀₀ₙₘ˜0.7-1. | |
| 3 | Pellet cells. | No concentrating step. |
| 4 | Resuspend cells in MS | Adjust media to 55 g/L |
| | medium + MES (pH 5.6) + | sucrose, 200 μM |
| | 20 g/L sucrose, 200 μM | cells to O.D.₆₀₀ₙₘ˜2.5 if >2.5. |
| | acetosyringone. Dilute | |
| | acetosyringone to | |
| | O.D.₆₀₀ₙₘ˜2.4. | |
| 5 | Incubate cells 1 hr; use in | Incubate cells one hour. |
| | 2-3 hours for infiltration | Add 0.005% Tween 20 before |
| | | infiltration. |

Pretreatment in Presence of High Sucrose

Construction of Plant Expression Vectors

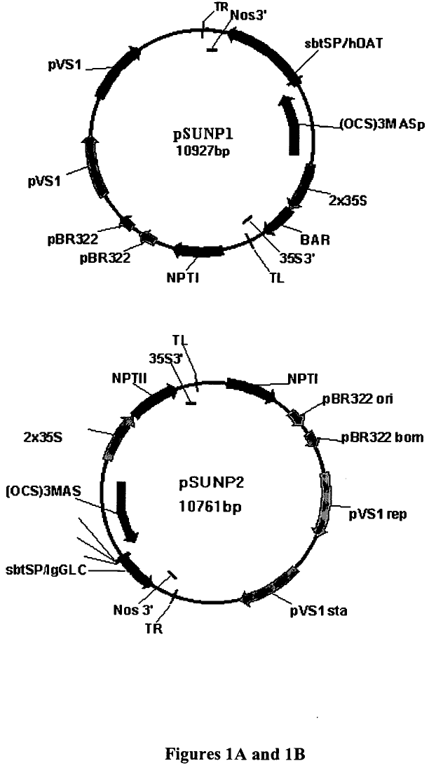

Plasmid vectors containing the gene of interest were constructed using standard molecular biology techniques. The basic elements include: a starting plasmid that is capable of replicating in both E. coli and Agrobacterium, the right and left T-DNA borders flanking the gene of interest driven by a promoter and with a targeting sequence. The necessary elements are assembled to produce the plasmids shown in FIGS. 1A and 1B.

FIG. 1A shows plasmid pSUNP1 comprising the 2,4-D inducible promoter (OCS)3Mas promoter (Gelvin et al., U.S. Pat. No. 5,955,646), driving the subtilisin secretion sequence (Janzik et al., Biol. Chem. 275: 5193-5199 (2000)), translationally fused to the heavy chain of anti-tissue factor antibody (IgG1). In addition the plasmid contains the selectable marker for kanamycin resistance and the BAR gene for bialphos resistance which has no utility in this transient expression system.

Plasmid pSUNP2, shown in FIG. 1B, is similar to pSUNP1 except the heavy chain of anti-tissue factor antibody has been replaced by the light chain (kappa) for the same antibody.

Transformation and Preparation of Agrobacterium

Agrobacterium tumifaciens C58/C1 cultures bearing desired binary vectors, were grown for 2 days at 28° C. in modified YEB media (6 g/L yeast extract, 5 g/L peptone, 5 g/L sucrose, 2 mM magnesium sulfate) with appropriate antibiotics (100 μg/mL of kanamycin, 15 μg/mL of rifampicin, 25 μg/mL of gentamycin) to select for the plasmid and the correct bacteria. This cultures were diluted 1:50 in modified YEB medium supplemented with antibiotics, 50 mM potassium-phosphate buffer, pH 5.6, 20 μM of acetosyringone and cultured approximately 18-24 hours until O.D._{600 nm} was approximately 2.5-3.5. If necessary, bacterial cells were diluted to an O.D._{600 nm} of 2.4 and supplemented with 55 g/L of sucrose and 200 μM acetosyringone (to give a maximum of 220 μM acetosyringone and 60 g/L of sucrose). The suspension was incubated for 1 hour at room temperature (about 22° C.); 100 μg of 2,4-D (2,4-dichlorophenoxyacetic acid, Sigma) and 0.005% Tween 20 is added prior to use.

By way of comparison, in the method described by Kapila, et al., supra (1977), the YEB medium is composed of 5 g/L beef extract, 1 g/L yeast extract, 5 g/L peptone, 5 g/L sucrose, 2 mM magnesium sulfate with appropriate antibiotics (100 μg/mL of kanamycin, 15 μg/mL of rifampicin, 25 μg/mL of gentamycin) to select for the plasmid. A starter culture was diluted 1:500 in YEB medium supplemented with antibiotics, 10 mM MES, pH 5.6, 20 μM of acetosyringone and cultured approximately 18-24 hours until O.D._{600 nm} was approximately 0.7-1. The culture was centrifuged to collect the cells and resuspended in Murashigi-Skoog medium (Kapila et al., supra (1997)) with 10 mM MES, pH 5.6, 20 g/L sucrose and 200 μM of acetosyringone to an O.D._{600 nm} of 2.4. The suspension was incubated for 1 hour at room temperature (about 22° C.); and 100 μg of 2,4-D.

Vacuum-Infiltration and Incubation of Treated Lettuce

The Agrobacterium suspensions were used directly for vacuum-infiltration. In the case where the light chain is on one plasmid and the heavy chain is on a second plasmid (dual vector system), two Agrobacterium cultures must be prepared and mixed in equal proportions prior to infiltration. In this example, the light chain vector encoded kappa light chain was from the anti-tissue factor antibody called hOAT and heavy chain vector encoded the IgG1 heavy chain of hOAT. A whole head of lettuce was immersed in 1.2 L of suspension in a 2 L beaker and placed in vacuum desiccator. A vacuum (equivalent to a 7 kPa) was applied for 20 minutes followed by vacuum shock from quick release of the vacuum. Lettuce heads were rinsed in water and incubated for 3-4 days at room temperature and 16 hours daylight in closed transparent boxes on wet paper towels. After 3-4 days the leaves were cut from the base and homogenized for protein extraction.

Extraction and Purification of Protein

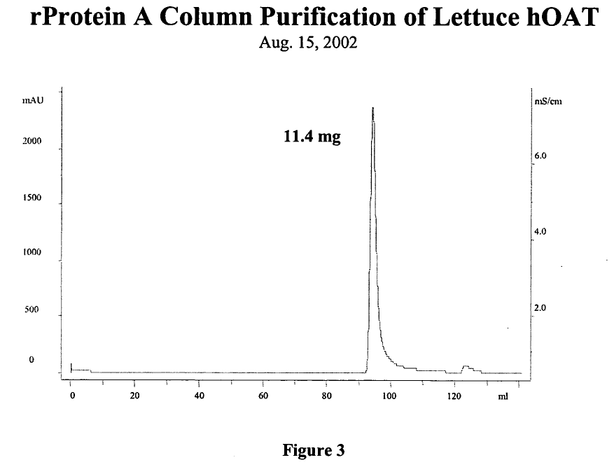

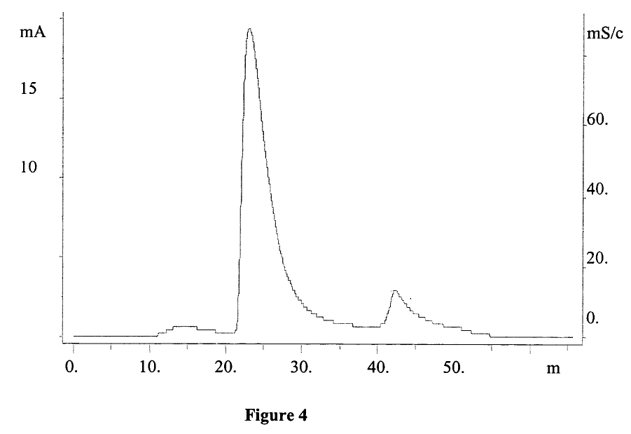

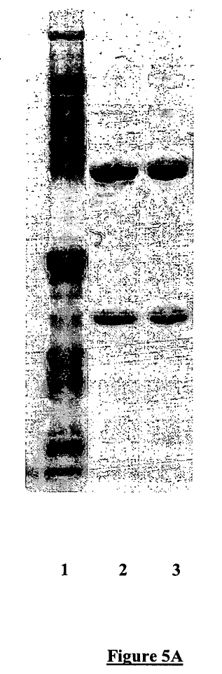

Protein was extracted in buffer (100 mM Tris-HCl, 5 mM EDTA, pH8.0, 1.5% insoluble PVP add before use) using equal part volume of buffer to weight of leaf. Leaves were homogenized in a Waring blender at high speed for 1 min and the resulting homogenate was centrifuged for 15 minutes at 10,000×g. Supernatant with non-precipitated cell debris was filtered through 12 layers of cheesecloth and centrifuged for 15 minutes at 20,000×g. Filtrate was loaded on a rProteinA sepharose fast flow affinity column (5 mL, resin from Amersham Pharmacia Biotech AB, Sweden) at 2 mL/min. Wash buffer and elution buffer used were 0.1 M Na-Acetate and 0.1 M Acetic acid, respectively. The protein was eluted with stepwise gradient pH method, i.e. 20% 0.1 M Acetic acid for 2 column volume, followed by 40%, 60% and 100% of 0.1 M Acetic acid for 4 column volume (FIG. 3). Peak fractions were collected and the pH was adjusted to 8.0 using 1M Tris-HCl, pH 8.0. Purified antibodies were quantified using O.D. at 280 nm. For further purification, Q Sepharose fast flow column (5 mL, resin from Amersham Pharmacia Biotech AB, Sweden) was equilibrated with 20 mM Tris-HCI pH 8.5 and ProteinA purified antibodies buffer exchanged into the same buffer were loaded onto the column. Antibody was eluted using salt stepwise elution method, i.e. 10% buffer of 20 mM Tris-HCl, pH8.0, 1M NaCl, for 2 column volume, followed by 50% of the buffer for 4 column volume and 100% of the buffer for 2 column volume. Peak fractions were collected and quantified by O.D. at 280 nm (FIG. 4). For buffer exchange the Millipore Ultrafree Centrifugal filter device 15 mL (Millipore Corporation, Bedford, Mass.) was soaked with 0.1 M NaOH for at least 1 hour. The buffer exchange for Q Sepharose Column purified antibodies was conducted with PBS to obtain greater than 1000× dilution. Buffer exchanged antibodies were filtered with Millex-GV 0.22 μm filter unit (Millipore Corporation, Bedford, Mass.) and quantified using O.D. at 280 nm.

The hOAT antibody was quantitated using an ELISA assay. To prepare one plate a coating solution of 5.5 μg or recombinant tissue factor (rTF) in 11 mL of coating buffer (35 mM NaHCO₃, 15 mM sodium bicarbonate, 50 mM NaCl, pH 9.0) is prepared. 100 μL of coating solution is transferred into each well and stored for up to 2 weeks covered at 4° C. The plate is washed 3-times with 400 μL of Wash Buffer (Kirkegaard&Perry) and 100 μL of sample is transferred to each well. The plate is incubated at room temperature for 1 hour with agitation then washed 6-times with Wash Buffer. Bound antibody is detected with Peroxidase-conjugated Donkey Anti-Human IgG (H+L) (Jackson ImmunoResearch) by incubating the plate at room temperature for 10 minutes followed by washing 6 times with Wash Buffer. ABTS substrate (BioFX) is added (100 μL) and after 10 minutes the reaction is stopped by the addition of 100 μL of 1% SDS. The absorbance at 405 nm is measured. A comparison of the level of expression from the Example 1 embodiment of the method and the Kapila method is shown in Table 2.

TABLE 2
Expression* of hOAT

| Method | Experiment 1 | Experiment 2 | Experiment 3 | Average |
|---|---|---|---|---|
| Kapila* | 2.4 | 6.0 | 3.2 | 3.9 |
| Example 1 | 27.0 | 17.6 | 12.5 | 19.0 |
| Results | | | | |

*The expression levels shown are in mg/kg-biomass and represent an average of three determinations from separate infiltrations in each experiment using a common lot of plant material.

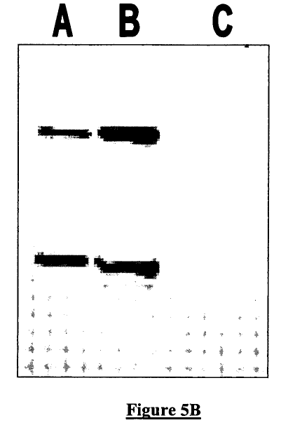

The SDS-PAGE was performed as described by Laemmli (1970) on 12% Tris Glycine minigels. The samples were prepared by mixing the protein extracts with a loading buffer (4:1, v/v) and subsequent heating at 70° C. for 10 minutes. Protein bands were detected by staining with Coomassie Blue (FIG. 5A) or electrophoretically transferred onto PVDF membrane. The membranes were blocked in 1:10 dilution of 2×PBS with 10% skim milk for one hour at room temperature. After washes, the blots were incubated for one hour at room temperature with anti-human IgG antibody conjugated with horseradish peroxidase (Binding site). FIG. 5B shows the IgG heavy and light chains detected by incubating the blots with enhanced chemiluminescence Western blotting detection reagents according to manufacturer's instructions (Pierce).

### Example 2

### Optimization of Sucrose Concentration

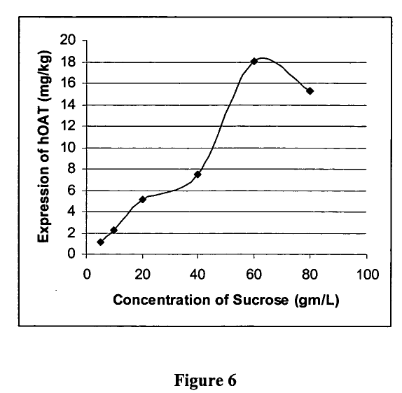

It was realized that the concentration of sucrose and the effect of osmotic shock due to the sucrose could be an important parameter for increasing expression. To test the effect of sucrose concentration on the level of protein production, the procedure of Example 1 was followed except a range of sucrose concentrations were tested for their effect on the expression of anti-tissue factor antibody. Based on the results from FIG. 6 a final sucrose concentration of 60 g/L was selected for the standard method.

### Example 3

### Optimization of Surfactant

To characterize the effect of surfactants the procedure as described in Example 1 was followed except the surfactant used was varied both in type and concentration.

TABLE 3
Effect of different surfactants on expression of hOAT

| | | Level of Expression |
|---|---|---|
| Detergent | Detergent Concentration | (mg/kg-biomass) |
| Tween 20 | 0.002% | 12.6 |
| Tween 20 | 0.005% | 16.0 |
| Tween 20 | 0.01% | 11.9 |
| Tween 80 | 0.005% | 13.2 |
| Nonidet NP40 | 0.005% | 9.6 |
| Triton X-100 | 0.005% | 15.0 |
| SDS | 0.005% | 1.3 |
| Silwet L-77 | 0.005% | 3.2 |
| Silwet L-77 | 0.02% | 14.5 |
| No Detergent | — | 3.5 |

Table 3 shows the results of a survey of different non-ionic and ionic detergents. The list is not meant to be exhaustive but shows the significant effect from varying this parameter. Tween-20 at 0.005% provides particularly high expression levels of heterologous protein.

### Example 4

### Effect of 2,4-D on Expression by (OCS)3MAS Promoter in Transient Expression System

A number of different promoters (5′ transcriptional regulatory regions) are available and commonly used for the expression of heterologous genes in plants. A literature survey and limited testing was done to determine a suitable promoter for this method. Table 4 shows the expression of hOAT from the synthetic promoter OCS3MAS using the chemical inducer 2,4-D. While it was known that the (OCS)3MAS promoter is induced by various factors most notably wounding, this study shows that transient delivery of the gene using Agrobacterium also results in inducible expression. This study was done using the original Kapila method (MS Method) for bacterial preparation and infiltration as described in Example 1 which is why the expression level is low than seen in some other examples. Based on this study a final concentration of 100 μg/mL 2,4-D was se

TABLE 4
Effect Of 2,4-D Concentration On hOAT Expression

| 2,4-D Concentration. (M) | Expression of hOAT (mg/kg) |
|---|---|
| 0 | 0.25 |
| 10 | 0.45 |
| 50 | 0.5 |
| 100 | 2.5 |
| 200 | 2.1 |

This limited survey is meant only as an example and it is possible that much higher expression could be obtained from anaerobically inducible promoters such as that from the Adh gene or from reportedly strong promoters of “housekeeping” genes such as eIF4 or from the small subunit of RUBISCO.

### Example 5

### Comparison of Dual Vectors vs. Bicistronic Vector for Transient Expression of hOAT

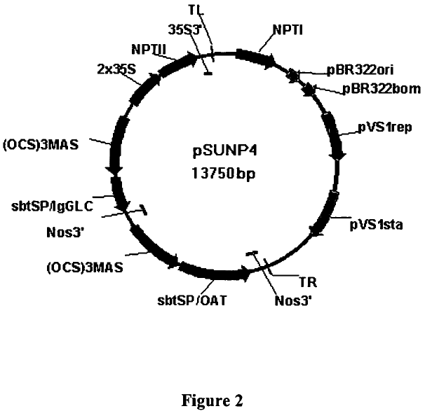

In this example, the use of the dual vector system (as described in Example 1) was compared to the expression of the heavy and light chains from a single vector (the bicistronic system). Plasmid pSUNP4 (FIG. 2) depicts a plasmid vector with both the heavy and light chains in the T-DNA region of the same plasmid. The (OCS)3MAS promoter is used to drive the expression of both the heavy and light chain of anti-tissue factor genes with the signal sequence coding for the subtilisin.

Lettuce was infiltrated with either two Agrobacterium cultures bearing the dual vectors or a single Agrobacterium culture bearing the bicistronic vector pSUNP4 and the levels of expression of hOAT determined as in Example 1. The results showing the levels of expression of hOAT using these two vector systems is shown in Table 5.

TABLE 5
Comparison Of hOAT Expression* Using Dual Vectors Or A
Bicistronic Vector.

| Vector System | Experiment 1 | Experiment 2 |
|---|---|---|
| Co-transient expression | 13.4 | 15.6 |
| Bi-cistronic expression | 25.9 | 27.6 |

*The expression levels shown are in mg/kg-biomass and represent an average of duplicate determinations from separate infiltrations in each experiment using a common lot of plant material.

### Example 6

### Transient Expression of Recombinant Antibody Against Shiga Toxin 2

In this example, it was demonstrated that transient expression can be used to produce another antibody. cαStx2 is a chimeric antibody that binds and neutralizes Shiga toxin 2 produced by enterohemorrhagic E. coli. The genes for the cαStx2 heavy and light chains were introduced into the dual vectors to generate the cαStx2 vectors that are similar to pSUNP1 and pSUNP2. These constructs and were transferred to Agrobacterium and used to agro-infiltrate lettuce, using the method as described in Example 1. After transient expression, crude extracts from plant cell as well as antibody obtained after Protein A purification were analyzed by ELISA.

The ELISA was performed by coating maxisorp 96-well plates with Stx2 antigen, which were covered with plastic film and stored at 4° C. until use. To measure antibody production, the wells were washed 3 times with buffer before using to remove the coating solution. Plant cell extract or purified protein solution was added to the coated wells. After 1 hour at room temperature, the wells were washed 3 times with buffer, and a dilution of an anti-human Kappa chain-HRP antibody was added. The plates were then incubated at room temperature for 1 hour and washed 3 times with wash buffer. To detect the presence of the probe antibody, ABTS substrate reagent was added and incubated for several minutes at room temperature, followed by ABTS quench buffer. Absorbance was read at 405 nm on an automatic plate reader.

ELISA analysis showed that samples with transiently expressed proteins contained functionally active cαStx2 antibody.

Those skilled in the art will recognize, or be able to ascertain, using no more than routine experimentation, numerous equivalents to the specific substances and procedures described herein. Such equivalents are considered to be within the scope of this invention, and are covered by the following claims.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

The patents, patent applications, international applications and references described herein and below are incorporated herein in their entireties.

### References

Artsaenko et al., Plant J. 8:745-50 (1995)

Banjoko et al., Plant Physiol. 107:1201-08 (1995)

Baum et al., Mol. Plant-Microbe Interact. 5:382-87 (1996)

Binet et al., Plant Science 79: 87-94 (1991)

Borisjuk et al., Nature Biotechnology 17:466-69 (1999)

Brown et al., Nucleic Acids Res. 17:8991 (1989)

Cabanes-Macheteau, et al., Glycobiology 9:365-72 (1999)

Christensen et al., Plant Molec. Biol. 12:619-632 (1989)

Christou, P. Plant Mol Biol. 35:197-203 (1996)

Conrad & Fiedler Plant Mol. Biol. 38:101-09 (1998)

Dennis et al., Nucleic Acids Res. 12:3983 (1984)

DeWilde et al., Plant Sci. 114:231-41 (1996)

During et al. Plant Molecular Biology 15:287-93 (1990)

What is claimed is:

1. A method for expressing a heterologous protein in a plant, comprising:
infiltrating plant tissue with Agrobacterium cells in the presence of a surfactant, the Agrobacterium cells comprising an expression construct capable of expressing a heterologous polypeptide in cells and/or intercellular spaces of the plant tissue; and
incubating the plant tissue under conditions suitable for expressing the polypeptide.

2. A method for expressing a heterologous protein in a plant, comprising:
infiltrating plant tissue with Agrobacterium cells, the Agrobacterium cells comprising an expression construct capable of expressing a heterologous polypeptide in plant cells and/or intercellular spaces of the plant tissue;
incubating the plant tissue under conditions suitable for expressing the polypeptide; and
isolating greater than about 1 mg of the polypeptide from a kilogram of treated plant tissue.

3. A method for expressing a heterologous protein in a plant, comprising:
infiltrating plant tissue with Agrobacterium cells, the Agrobacterium cells comprising an expression construct capable of expressing a heterologous polypeptide in plant cells and/or intercellular spaces of the plant tissue; and
incubating the plant tissue under conditions suitable for expressing the polypeptide,
wherein the plant tissue is obtained from a plant at least about a week post-harvest.

4. A method for expressing a heterologous protein in a plant, comprising:
culturing Agrobacterium cells comprising an expression construct capable of expressing a heterologous polypeptide in the plant cells and/or intercellular spaces of the plant tissue, in a culture medium;
directly contacting the culture medium comprising the Agrobacterium cells with plant tissue with or without a dilution step;
infiltrating the plant cells with the culture medium comprising the Agrobacterium cells; and
incubating the plant tissue under conditions suitable for expressing the polypeptide.

5. The method according to any of claims 1-3 and 5, wherein the heterologous polypeptide is isolated from the plant tissue.

6. The method according to any of claims 1-5, wherein the vector comprises at least one T-DNA border.

7. The method according to any of claims 1-5, further comprising the step of introducing the vector into at least one Agrobacterium cell and culturing the cell until sufficient quantities of cells are obtained for infiltrating the plant tissue.

8. The method of any of claims 1-5, in which the plant is selected from the group consisting of: lettuce, alfalfa, mung bean, spinach, dandelion, radicchio, arugula, endive, escarole, chicory, artichoke, maize, potato, rice, soybean, Crucifera, duckweed, maize, potato, rice, soybean, spinach, tomato and tobacco.

9. The method according to claim 8, wherein the Crucifera plant comprises Brassica or Arabidopsis.

10. The method of any of claims 1-4 in which the Agrobacterium is cultured overnight and diluted to an absorbance at 600 nm of about 2.5 before use.

11. The method of any of claims 1-4, in which the Agrobacterium is infiltrated using vacuum.

12. The method of any of claims 2-4, in which the infiltration is done in the presence of a surfactant.

13. The method of claim 1 in which the surfactant is selected from the group comprising Tween-20, Tween-80, Triton X-100, NP-40, Silwet L-77.

14. The method of claim 12, in which the surfactant is selected from the group comprising Tween-20, Tween-80, Triton X-100, NP-40, Silwet L-77.

15. The method of claim 1 in which the surfactant is about 0.005% Tween-20.

16. The method of claim 12 in which the surfactant is about 0.005% Tween-20.

17. The method of any of claims 1-5 in which the infiltration is performed in the presence of an agent for inducing osmotic shock.

18. The method of claim 17, in which the agent for inducing osmotic shock is sucrose.

19. The method of claim 18, in which the sucrose is at a concentration of about 60 g/L.

20. The method of any of claims 1-4 in which the protein is collected by grinding whole plants or leaves.

21. The method of any of claims 1-4 in which the protein is collected from the intercellular fluid of the plant.

22. The method of any of claims 1-4, in which the protein is targeted to the endoplasmic reticulum of the plant cell.

23. The method of any of claims 1-4 in which multiple genes are delivered to the plant by Agrobacterium.

24. The method of any of claims 1-4, in which a plurality of strains of Agrobacterium are combined and co-infiltrated into the desired plant

25. The method of claim 24, wherein multiple genes are delivered by the Agrobacterium strains.

26. The method of claim 23 in which the multiple genes encode proteins that assemble to form a multi-subunit protein.

27. The method of any of claims 1-4, in which the protein expressed comprises a protein in the immunoglobulin superfamily.

28. The method of claim 27, wherein the protein is an antibody, T cell receptor and Major Histocompatibility Complexes, or biologically functional fragments or single chain derivatives thereof.

29. The method of claim 25, in which the multiple genes encode proteins comprising members of a pathway.

30. The method of claim 29, wherein the pathway is a chemical synthesis pathway or a signaling pathway.

31. The method of any of claims 1-4, in which the protein is glycosylated.

32. The method of any of clams 1-4, further comprising the step obtaining the expression construct which has been expressed and stably introducing it into a plant.

33. The method of any of claims 1-4, wherein the plant tissue is from a transgenic plant.

34. A method of screening for mutations or variant sequences, comprising: performing the method of any of claims 1-4 in a plurality of plant tissue samples with a plurality of expression constructs, the expression constructs comprising encoding sequences which are at least about 50% identical and which differ from one another by the presence of one or more mutations and/or which encode one or more variant amino acids.

35. The method of claim 34, wherein the one or more mutations comprises a deletion, insertion, substitution or rearrangement.

36. The method of claim 34, wherein expression constructs are identified which encode heterologous polypeptides having an improved property compared to heterologous polypeptides which do not comprise the one or more mutations and which do not encode variant amino acid sequences.

37. The method of claim 36, wherein the improved property comprises increased activity and/or stability.

38. The method of claim 37, wherein the increased activity comprises increased binding affinity of a heterologous protein for a binding partner which specifically binds to the heterologous protein.

39. The method of claim 38, wherein the protein comprises a member of immunoglobulin superfamily.

40. The method of claim 39, wherein the protein is selected from the group consisting of: antibodies, T cell receptors and Major Histocompatibility Complexes, or biologically functional fragments and single chain derivatives thereof.

41. The method of claim 33, wherein the one or more mutations comprise insertion of human sequences into a heterologous encoding sequence from a non-human animal.
